(19) United States
(12) Patent Application Publication
(10) Pub. No.: US 2016/0018339 A1
(43) Pub. Date: Jan. 21, 2016

(54) AUTONOMOUS REMOTE SENSOR FOR DETERMINING A PROPERTY OF A FLUID IN A BODY OF WATER

(71) Applicant: Halliburton Energy Services, Inc., Houston, TX (US)

(72) Inventor: David L. PERKINS, Houston, TX (US)

(21) Appl. No.: 14/771,569

(22) PCT Filed: Apr. 2, 2013
(86) PCT No.: PCT/US2013/034900

§ 371 Date: Aug. 31, 2015

(51) Int. Cl. G01N 21/85; G01N 21/27; G01N 33/18

(52) U.S. Cl. CPC G01N 21/8507; G01N 33/1886; G01N 33/1833; G01N 21/27

(57) ABSTRACT

An autonomous remote sensor for analyzing a fluid in a body of water comprises: a vessel, wherein the vessel moves through the body of water; and an analyzer, wherein the analyzer: (A) is located on or adjacent to the vessel; (B) incorporates one or more Integrated Computational Elements (ICE); and (C) is capable of determining at least one property of the fluid by at least contacting the fluid with radiated energy and detecting the interaction between the radiated energy and the fluid. A method of analyzing a fluid in a body of water comprises: providing a vessel, wherein the vessel moves through the body of water; and determining at least one property of the fluid using the analyzer. The analyzer can also have a spectral resolution less than 4 nm.

### TECHNICAL FIELD

Vessels are used to navigate through bodies of water. Analyzers are used to determine one or more properties of a fluid. An analyzer can be positioned on a vessel in order to determine a property of a fluid in the body of water. Common analyzers include spectrometers. The analysis of the fluid in the body of water can be used to discover an oil or gas reservoir, the source of leaks in well equipment, or the boundaries of an oil slick.

### SUMMARY

According to an embodiment, a method of analyzing a fluid in a body of water comprises: providing a vessel, wherein the vessel moves through the body of water; and determining at least one property of the fluid using an analyzer, wherein the step of determining comprises: contacting the fluid with radiated energy; and detecting the interaction between the radiated energy and the fluid, wherein the analyzer is located on or adjacent to the vessel, and wherein the analyzer incorporates one or more integrated computational elements (ICEs).

According to another embodiment, an autonomous remote sensor for analyzing a fluid in a body of water comprises: a vessel, wherein the vessel moves through the body of water; and an analyzer, wherein the analyzer: (A) is located on or adjacent to the vessel; (B) incorporates one or more ICEs; and (C) is capable of determining at least one property of the fluid by at least contacting the fluid with radiated energy and detecting the interaction between the radiated energy and the fluid.

According to another embodiment, a method of analyzing a fluid in a body of water comprises: providing a vessel, wherein the vessel moves through the body of water; and determining at least one property of the fluid using an analyzer, wherein the step of determining comprises: contacting the fluid with radiated energy; and detecting the interaction between the radiated energy and the fluid, wherein the analyzer is located on or adjacent to the vessel, and wherein the analyzer has a spectral resolution less than 4 nm.

### BRIEF DESCRIPTION OF THE FIGURES

The features and advantages of certain embodiments will be more readily appreciated when considered in conjunction with the accompanying figures. The figures are not to be construed as limiting any of the preferred embodiments.

FIG. 1 depicts a vessel and an analyzer according to an embodiment wherein the vessel moves through the water on the surface of the water.

FIG. 2 depicts the vessel and the analyzer according to another embodiment wherein the vessel moves through the water beneath the surface of the water.

FIG. 3 depicts the analyzer according to certain embodiments.

FIG. 4 is a side elevation sectional view of an illustrative representative Integrated Computational Element (ICE) construction.

FIGS. 5 and 6 are graphs illustrating respective wavelength dependent transmission light intensity through and reflectance light intensity from multilayered ICE.

FIG. 7 is a sectional elevation view of a tube in which a fluid is flowing during analysis using an illustrative representative ICE calculation device analyzer.

### DETAILED DESCRIPTION

As used herein, the words “comprise,” “have,” “include,” and all grammatical variations thereof are each intended to have an open, non-limiting meaning that does not exclude additional elements or steps.

As used herein, a “fluid” is a substance having a phase that tends to flow and to conform to the outline of its container when the substance is tested at a temperature of 71° F. (22° C.) and a pressure of one atmosphere “atm” (0.1 megapascals “MPa”). A fluid can be a liquid or gas. A fluid can have only one phase or more than one phase. In the oil and gas industry, a fluid having only one phase is commonly referred to as a single-phase fluid and a fluid having more than one phase is commonly referred to as a multi-phase fluid. A colloid is an example of a multi-phase fluid. A colloid can be: a slurry, which includes a continuous liquid phase and undissolved solid particles as the dispersed phase; an emulsion, which includes a continuous liquid phase and at least one dispersed phase of immiscible liquid droplets; a foam, which includes a continuous liquid phase and a gas as the dispersed phase; or a mist, which includes a continuous gas phase and liquid droplets as the dispersed phase.

Oil and gas hydrocarbons are naturally occurring in some subterranean formations. A subterranean formation containing oil or gas is sometimes referred to as a reservoir. A reservoir may be located under land or off shore. Reservoirs are typically located in the range of a few hundred feet (shallow reservoirs) to a few tens of thousands of feet (ultra-deep reservoirs). In off-shore drilling, production tubing is inserted into a body of water and extends through the water to the floor of the body of water. A wellbore is then drilled from the floor through the sub-water land into a reservoir or adjacent to a reservoir. The floor is the surface of the sub-water land. The wellhead is located at or near the top of the floor. The body of water and the wellbore can be several hundred to several thousands of feet deep. As used herein, the term “body of water” includes, without limitation, either formed by nature or man-made, a river, a pond, a lake, a gulf, a canal, a reservoir, a retention pond, or an ocean. As used herein, the term “water” means the water located within the body of water. The water can be freshwater, salt water, effluent, produced or flowback water, or brackish water.

It is often desirable to analyze a fluid in a body of water in order to determine one or more properties of the fluid. For example, some reservoirs may naturally eject oil or gas into the water from the floor of the body of water. During oil or gas exploration, a vessel, such as a submersible can move through the body of water near the floor. An analyzer can be included on the vessel and analyze the water as the vessel moves through the water. The analyzer detects and quantifies the property or properties of interest in the fluid, while simultaneously determining and recording the position of the vessel. The position dependent data collected by the analyzer can be used to determine the presence and location of a hydrocarbon reservoir.

In another application, it may be desirable to analyze the fluid in a body of water to test for leaks in equipment used in the exploration and production of hydrocarbons within a reservoir. For example, in off-shore drilling, one or more tubing strings extend from the rig floor through the body of water to the wellhead and into the subterranean formation. A leak can occur at one or more locations at or near the wellhead or along the length of the tubing strings in the water. The analysis of the water near the well equipment can be used to determine the presence and location of leaks, and also possibly the quantification or concentration of the liquids leaking into the water.

Yet another example is when an oil slick occurs. An oil slick can occur when oil contaminates a body of water. During off-shore oil production, a component of the drilling rig or production tubing may become damaged to such an extent that oil may no longer be contained in the system, but rather, may flow into the surrounding water. A vessel, for example a boat, and the analyzer can be used to determine the boundaries of the oil slick, above and beneath the surface, and possibly the concentration of contaminates in the water.

As used herein, the term “oil” means a liquid comprising a hydrocarbon when measured at a temperature of 71° F. (21.7° C.) and a pressure of one atmosphere. Examples of oil include, but are not limited to: crude oil; a fractional distillate of crude oil; a fatty derivative of an acid, an ester, an ether, an alcohol, an amine, an amide, or an imide; a saturated hydrocarbon; an unsaturated hydrocarbon; a branched hydrocarbon; a cyclic hydrocarbon; and any combination thereof. Crude oil can be separated into fractional distillates based on the boiling point of the fractions in the crude oil. An example of a suitable fractional distillate of crude oil is diesel oil. The saturated hydrocarbon can be an alkane or paraffin. The paraffin can be an isoalkane (isoparaffin), a linear alkane (paraffin), or a cyclic alkane (cycloparaffin). The unsaturated hydrocarbon can be an alkene, alkyne, or aromatic. The alkene can be an isoalkene, linear alkene, or cyclic alkene. The linear alkene can be a linear alpha olefin or an internal olefin.

There are several devices that can be used to analyze a fluid sample. A spectrometer is an example of a device that can be used to analyze a fluid sample. Spectroscopy is the study of the interaction between matter and radiated energy. Generally, an energy source, such as light, is directed to a sample. A detector can then detect the light after the light interacts with the sample. Light can interact with a sample when it is transmitted through, reflected from, emitted from, or scattered from a sample. Spectroscopic data is often represented by a spectrum, which is a plot of a detectors response as a function of wavelength or frequency.

Spectroscopic techniques that employ electromagnetic radiation are typically classified by the wavelength region of the spectrum and include radio, microwave, terahertz, far infrared, near infrared, visible, ultraviolet, x-ray and gamma spectroscopy. A wavelength is the distance over which a wave repeats itself, is inversely proportional to the frequency, and is reported in units of length (e.g., micrometers, nanometers, or meters). The higher the frequency the shorter the wavelength and the lower the frequency the longer the wavelength. The frequency is the number of occurrences per unit of time, reported in units of seconds. A wavenumber or the number of waves per unit distance, is proportional to the reciprocal of the wavelength, reported in units of inverse meters (m⁻¹) or inverse centimeters (cm⁻¹). The wavelength regions for each type of electromagnetic radiation are different. For example, the near infrared region has a wavelength range of approximately 800 nanometers (nm) to 2,500 nm; whereas, the ultraviolet region has a wavelength range of approximately 10 nm to 350 nm.

Types of spectroscopy can also be distinguished by the nature of the interaction between the energy and the material. These interactions include absorption, emission, elastic scattering and reflection, impedance, inelastic scattering, and coherent interactions. Absorption occurs as a portion of the incident radiation is absorbed by the material. Absorption is typically expressed as transmittance or the ratio of the intensity of electromagnetic radiation that is transmitted through the sample to the incident electromagnetic radiation's intensity. Emission indicates that radiative energy is released by the material. A material's blackbody spectrum is a spontaneous emission spectrum determined by its temperature. Emission can be induced by electromagnetic radiation in the case of fluorescence. Elastic scattering and reflection spectroscopy determine how incident radiation is reflected or scattered by a material. Impedance spectroscopy studies the ability of a medium to impede or slow the transmittance of energy. Inelastic scattering involves an exchange of energy between the radiation and the matter that shifts the wavelength of the scattered radiation. These include Raman and Compton scattering. Coherent or resonance spectroscopies are techniques where the radiative energy couples two quantum states of the material in a coherent interaction that is sustained by the radiating field. The coherence can be disrupted by other interactions, such as particle collisions and energy transfer, and thus, often require high intensity radiation to be sustained. Nuclear magnetic resonance (NMR) spectroscopy is a widely used resonance method and ultrafast laser methods are also now possible in the infrared and visible spectral regions.

The spectral resolution of spectrometers can vary widely. The spectral resolution (R), reported in units of meters or nanometers (nm), is a measure of the spectrometer's ability to resolve features in the electromagnetic spectrum and can be calculated as follows:

$$R = \frac{λ}{Δ λ}$$

where Δλ is the smallest difference in wavelengths that can be distinguished at a wavelength of λ. The higher the spectral resolution of a spectrometer, the lower the value of R will be. For example, a spectrometer that has a resolution of 10 nanometers (nm) has a higher resolution compared to another spectrometer with a resolution of 50 nm. However, the observed signal (Sₒ) of the spectrometer is not solely dependent on the spectral resolution of the spectrometer but it is also dependent on the line width of the signal (Sᵣ). Therefore, the observed resolution is the convolution of the two sources and can be calculated as follows:

So(λ)=Sr(λ)*R(λ)

When the signal line width is significantly greater than the spectral resolution, the effect can be ignored and one can assume that the measured resolution is the same as the signal resolution. Conversely, when the signal line width is significantly narrower than the spectral resolution, the observed spectrum will be limited solely by the spectral resolution.

Currently, there are some analyzers used to analyze a fluid in a body of water. However, these analyzers do not possess the desired spectral resolution. When using a spectrometer in the aforementioned applications, it is desirable to have the highest spectral resolution as possible. Having a higher spectral resolution allows for more accurate analysis. Therefore, there exists a need for an analyzer having a desired spectral resolution to analyze a fluid in a body of water in order to determine the presence of a reservoir, determine leaks in well equipment, and/or determine the presence or boundaries of an oil slick.

According to an embodiment, a method of analyzing a fluid in a body of water comprises: providing a vessel, wherein the vessel moves through the body of water; and determining at least one property of the fluid using an analyzer, wherein the step of determining comprises: contacting the fluid with radiated energy; and detecting the interaction between the radiated energy and the fluid, wherein the analyzer is located on or adjacent to the vessel, and wherein the analyzer incorporates one or more integrated computational elements (ICEs).

According to another embodiment, an autonomous remote sensor for analyzing a fluid in a body of water comprises: a vessel, wherein the vessel moves through the body of water; and an analyzer, wherein the analyzer: (A) is located on or adjacent to the vessel; (B) incorporates one or more ICEs; and (C) is capable of determining at least one property of the fluid by at least contacting the fluid with radiated energy and detecting the interaction between the radiated energy and the fluid.

According to another embodiment, a method of analyzing a fluid in a body of water comprises: providing a vessel, wherein the vessel moves through the body of water; and determining at least one property of the fluid using an analyzer, wherein the step of determining comprises: contacting the fluid with radiated energy; and detecting the interaction between the radiated energy and the fluid, wherein the analyzer is located on or adjacent to the vessel, and wherein the analyzer has a spectral resolution less than 4 nm.

Any discussion of the embodiments regarding the analysis of the fluid is intended to apply to all of the method embodiments and apparatus embodiments. Any discussion of a particular component of an embodiment (e.g., an analyzer) is meant to include the singular form of the component as well as the plural form of the component, without continually referring to the component in both the singular and plural form throughout. For example, if a discussion involves “the analyzer 24,” it is to be understood that the discussion pertains to one analyzer (singular) and two or more analyzers (plural).

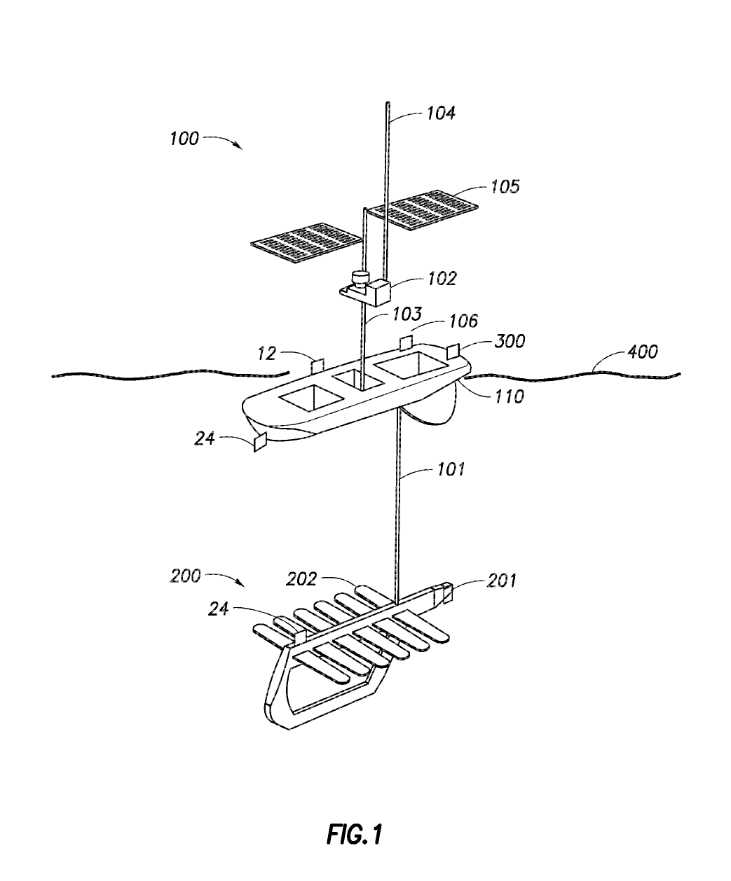

Turning to the Figures. FIG. 1 depicts a vessel 100 according to an embodiment. The autonomous remote sensor includes the vessel 100. The vessel 100 moves through the body of water. As shown in FIG. 1, the vessel 100 can include a boat 110. The boat 110 can move through the body of water at the surface of the water 400.

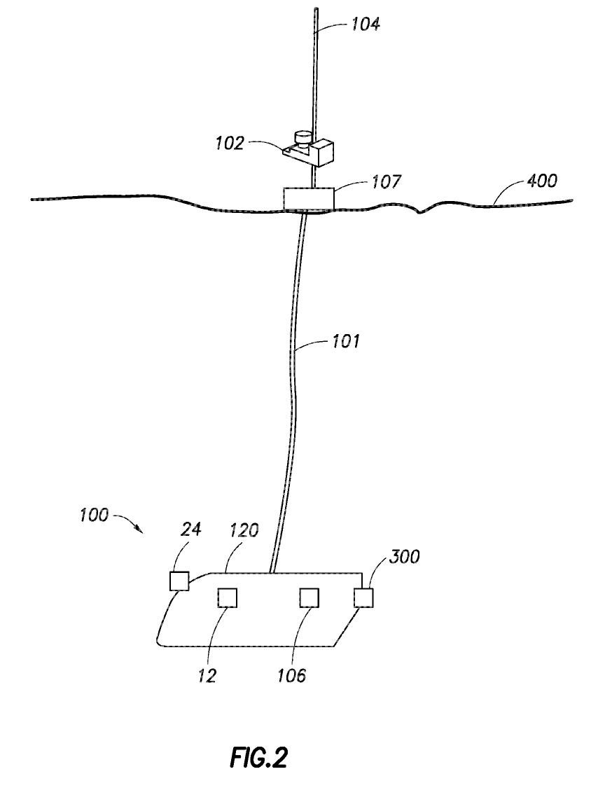

FIG. 2 depicts the vessel 100 according to another embodiment. The vessel 100 can include a submersible 120. The submersible 120 can move through the body of water at a specified distance below the surface of the water 400. For example, the submersible 120 can be designed such that it can move through the body of water at a depth ranging from 10 feet to several hundreds of feet below the surface. In this manner, the submersible 120 can navigate through the body of water at one or more desirable depths. The desired depth can depend on the area of water to be analyzed (e.g., at the wellhead, at one or more connections of the tubing making up a tubing string, or at the floor for oil or gas exploration). The depth can therefore depend on the desired operation of the vessel and the analyzer.

The vessel 100 can further include a navigation system. According to an embodiment, the navigation system can include a transmitter and receiver module 102. The transmitter and receiver module 102 can be used in conjunction with a global positioning satellite (GPS) system. The transmitter and receiver module 102 can communicate with the GPS system to determine the location of the vessel continuously and relay that information to a remote location. The vessel 100 can also include one or more antennae 104. The antenna 104 can also be used in communicating with the GPS system, another transmitter, or another receiver. The transmitter and receiver module 102 can also be used to receive coordinate instructions from an operator. The operator can be, for example, on another vessel, an off-shore platform, or on land. The transmitter and receiver module 102 can transmit the vessel's location based on information from the GPS system to a receiver (not shown). The transmitter and receiver module 102 can also receive navigation instructions from a transmitter (not shown). According to this embodiment, the transmitter and receiver module 102 of the vessel 100 can transmit the vessel's location to the receiver, the operator can determine if the vessel's course needs to be altered, the operator can then alter the vessel's course by transmitting new navigation instructions to the transmitter and receiver module 102 of the vessel 100 from the transmitter. The operator can also completely guide the vessel 100 through the body of water during operations via the transmitter and receiver module 102, the transmitter and receiver (not shown), and optionally the GPS system.

According to another embodiment, the vessel 100 further comprises an on-board computer 106. The on-board computer 106 can be used to navigate the vessel 100 through the body of water. For example, a course for the vessel 100 can be pre-programmed into the on-board computer 106. The transmitter and receiver module 102 can communicate with GPS system such that the vessel 100 follows the pre-programmed course through the body of water. Of course, if the situation arises such that the course needs to be altered, then an operator can send new navigation instructions to the vessel 100 via the transmitter and receiver module 102 and optionally the antenna 104. It may be beneficial to include an override feature for the on-board computer 106 in the event it does become necessary to alter the vessel's course.

According to yet another embodiment, the vessel 100 can move through the body of water based on the analysis of the fluid of the body of water. A more detailed description of the analyzer 24 is discussed below. By way of example, if the vessel 100 is used to determine the presence of oil in body of water, then the analyzer 24 can be used to determine if the property of oil is present in the fluid being analyzed. If oil is not present, then the vessel 100 can move in a specific direction. However, once the analyzer determines the presence of oil in the fluid, then the vessel 100 can automatically move in a different direction. The vessel 100 can continue to move in this different direction so long as the analyzer 24 continues to determine the presence of the oil. If however, oil is no longer present in the fluid, or if the concentration of oil decreases below a specified value, then the vessel 100 can automatically alter its course based on the data from the analyzer 24. The direction and course the vessel 100 takes can be pre-programmed into the on-board computer 106 such that the data from the analyzer 24 and the property of interest directs the vessel 100 to change directions automatically. This embodiment can be useful in many different circumstances. For example, it may be desirable to pre-program the vessel 100 to navigate through the body of water near a wellhead or other well equipment. The vessel 100 can continuously move in a pre-programmed direction or course until the analyzer 24 detects the presence of a pre-determined property. Then, the vessel 100 can change direction or course in order to pinpoint the source of the leak.

The vessel 100 can further include a device for moving the vessel through the body of water. The device can be a self-propulsion system 200 or a propulsion device 300. The self-propulsion system 200 can include a rudder 201 and propulsion arms 202. The self-propulsion system 200 can be designed such that a movement of the water, for example via waves, can cause the boat 110 to move on the surface of the water 400. According to this embodiment, the boat 110 can move via the self-propulsion system 200 without any action on an operator's part. Thus, the system can be designed to work autonomously to move the boat 110 through the water so long as movement of the water exists. The propulsion arms 202 can be articulated such that the arms can move up and down and angle. The action of the arms can cause the boat 110 to move forward through the water. The rudder 201 can be used to move the boat 110 in a given direction.

The device for moving the vessel 100 through the body of water can also be a propulsion device 300. An example of a propulsion device 300 is a motor or engine and a propeller or impeller. As can be seen in FIGS. 1 and 2, the propulsion device 300 can be located at the stern of the boat 110 or submersible 120, typically positioned at the vessel's centerline, although it need not be restricted to the stern. The boat 110 or submersible 120 can also include a means for controlling the direction of the boat or submersible. For example, the means for controlling the direction can be a rudder (not shown) or one or more directional thrusters.

The vessel 100 can further include an umbilical 101. The umbilical 101 can extend down from the boat 110 and into the body of water. As shown in FIG. 1, the umbilical 101 can connect the boat 110 with the self-propulsion system 200. As shown in FIG. 2, the umbilical 101 can extend up from the submersible 120. According to this embodiment, the top of the umbilical 101 can be connected to one or more floatation devices 107. The floatation device 107 can be used to help orient the top of the umbilical 101 at the surface of the water 400. The transmitter and receiver module 102 and the antenna 104 (if included in the vessel) can be located on top of the floatation device 107. The umbilical 101 can include one or more data transmission wires (e.g., copper wires or optical fibers). The data transmission wires can be used to send data to the transmitter and receiver module 102 regarding the submersible's location or the data from the analyzer 24. The data transmission wires can also be used to relay information, for example new navigation instructions, from the transmitter and receiver module 102 to the on-board computer 106 or the analyzer 24. The umbilical 101 can also contain a coating or sheath, which can protect (physically or chemically) the data transmission wires from being adversely affected by the body of water. The length of the umbilical 101 can vary. Some of the factors affecting the desired length of the umbilical 101 include, but are not limited to, the desired depth of the analyzer, the preferred depth of the self-propulsion system 200, and the preferred depth of the submersible 120. In some instances for a submersible, the desired depth of the submersible and analyzer may render the use of an umbilical impractical or impossible. In these instances, data can be stored on board the submersible for retrieval at a later date or the data can be transmitted via acoustic or low frequency radio signals.

The vessel 100 can further comprise a power supply. The power supply can provide power to any or all of the following: the analyzer 24; the on-board computer 106; the propulsion device 300; the transmitter and receiver module 102; and any other device not specifically mentioned that requires power to operate. As can be seen in FIG. 1, the power supply can be solar panels 105. The power supply can also be one or more batteries, a wave driven power supply, or a power generator. The vessel 100 can include both, the solar panels and batteries, a wave driven power supply, a thermal-electric generator, or a power generator. Although not depicted in FIG. 2, solar panels 105 can also be located on top of the floatation device 107, wherein the power is transmitted from the solar panels 105 down to the components of the submersible 120 requiring power via the umbilical 101. Some of the components of the vessel 100, for example, the transmitter and receiver module 102 and/or the solar panels 105 can be positioned on the vessel 100 via one or more supports 103.

The autonomous remote sensor includes the vessel 100 and an analyzer 24, wherein the analyzer is located on or adjacent to the vessel. As shown in FIG. 1, the analyzer 24 can be located on the boat 110. Preferably, the analyzer is located on the boat at a position such that the fluid from the body of water can flow into a testing chamber of the analyzer. According to this example, the analyzer 24 can be located at the bow of the boat 110. The analyzer can also be located underneath the bottom of the boat depending on whether the fluid being analyzed should be at the surface of the water 400, in the case of an oil slick, or a few inches below the surface of the water. According to another embodiment, also shown in FIG. 1, the analyzer 24 can be located adjacent to the vessel 100, for example, on a component of the self-propulsion system 200. As shown in FIG. 2, the analyzer 24 can be located on the submersible 120. Depending on the desired depth of the analyzer, the analyzer can also be positioned at any location on the umbilical 101.

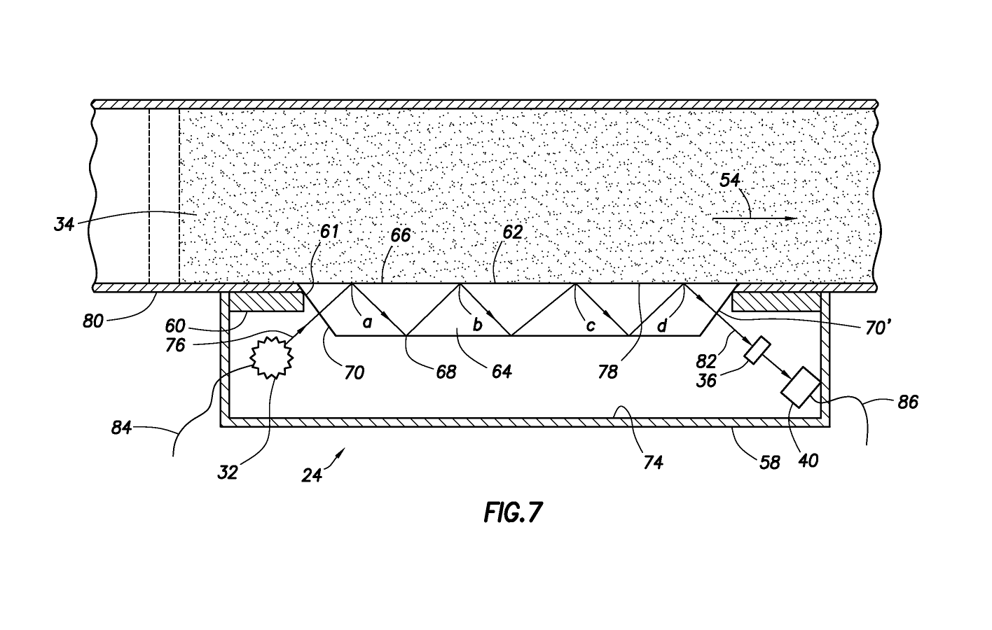

As shown in FIG. 7, the analyzer 24 can be located adjacent to a tube 80. The opening of the tube 80 can be positioned on part of the vessel 100 (e.g., the boat, the submersible, or the umbilical), such that the fluid 34 of the body of water flows into the tube. The opening of the tube can be positioned at the surface of the water 400 or a desired depth in the body of water, depending on the desired location of analysis. In this manner, the fluid 34 can flow through the tube 80 wherein the analyzer 24 can determine at least one property of the fluid. The autonomous remote sensor can further include a pump (not shown). The analyzer 24 can analyze the fluid 34 during fluid flow or when static (i.e., not flowing). For static testing, the pump can be used to direct the fluid 34 into a testing chamber, wherein once the fluid is located in the testing chamber, the fluid can be analyzed. Accordingly, the pump can be designed to cycle on and off at a desired time interval. Once the fluid is located in the testing chamber, the fluid can be analyzed and then the pump can cycle on to pump the fluid out of the testing chamber and pump new fluid in. For fluid flow testing, the pump can be designed such that it does not cycle off, but rather continuously pumps fluid through the tube 80 wherein analysis is performed in the fluid in the tube or continuously pumps the fluid into and out of a testing chamber. The sensor can also be designed such that a pump is not required, but rather the fluid 34 can flow into and through the tube 80 via the movement of the vessel 100 through the body of water. The movement of the vessel will direct the fluid into the tube and possibly into and out of the testing chamber in a continuous manner so long as there is movement of the vessel through the body of water.

According to an embodiment, the analyzer 24 has a spectral resolution of less than 4 nanometers (nm). According to another embodiment, the analyzer is a high-resolution spectrometer. According to another embodiment, the analyzer 24 has a spectral resolution of less than 2 nm, preferably less than 1 nm. Many factors can affect the spectral resolution of the analyzer. Some of the factors include, but are not limited to: the slit; the type of grating (e.g., ruled or holographic); for a ruled grating, the groove frequency; the type of detector and material the detector is made from; the amount of noise of the detector; and the configuration of the optical bench. One of ordinary skill in the art will be able to select the type of analyzer, the components and configurations thereof, and other parameters in order for the analyzer to have the specified spectral resolution.

The analyzer 24 is capable of determining at least one property of the fluid. The fluid 34 is from a body of water. The fluid 34 can be a colloid, for example, a slurry, an emulsion, or a foam. The fluid 34 can contain the water from the body of water and at least one other fluid and/or undissolved solids. The fluid 34 can contain plankton and or bacteria. The water of the body of water can be freshwater, brackish water, salt water, effluent, produced, or flowback water.

The at least one property of the fluid 34 can be selected from the group consisting of: asphaltenes; saturates; resins; aromatics; solid particulate content; hydrocarbon composition and content; gas composition C₁-C₁₃ and content; carbon dioxide gas; hydrogen sulfide gas; and correlated pressure, volume, or temperature properties including fluid compressibility, gas-to-oil ratio, bubble point, density, capacitance, resistivity, a petroleum formation factor, viscosity, a gas component of a gas phase of a petroleum, total stream percentage of water, gas, oil, solid particles, solid types, oil finger printing, reservoir continuity, and oil type; plankton and/or bacteria count and type; water elements including ion composition and content, anions, cations, salinity, organics, pH, mixing ratios, tracer components, contamination; or other hydrocarbon, gas, solids, or water properties that can be related to spectral characteristics, including the use of regression methods.

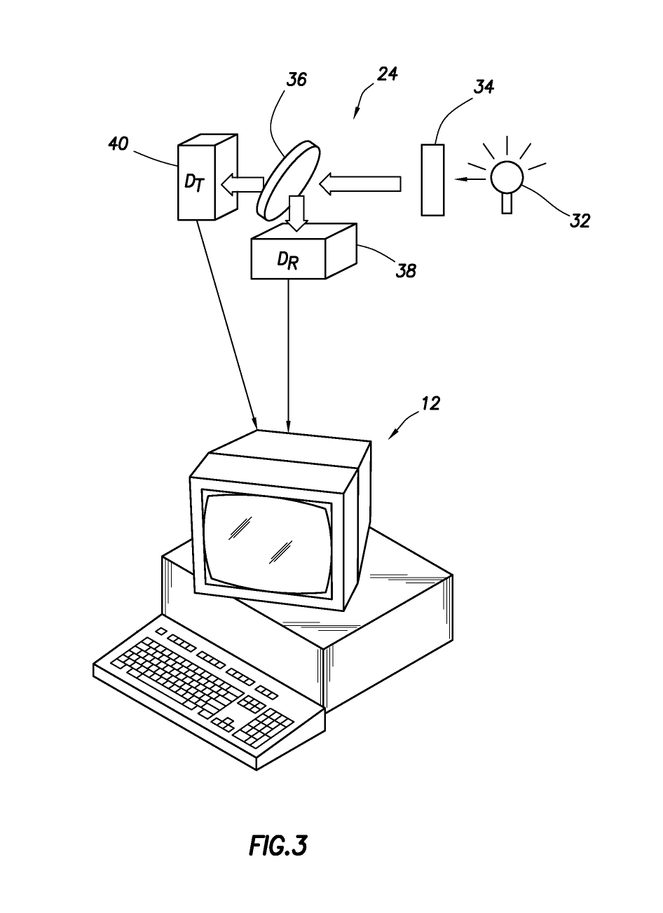

The at least one property of the fluid 34 is determined using the analyzer 24, wherein the at least one property is determined by at least contacting the fluid 34 with radiated energy and detecting the interaction between the radiated energy and the fluid. The analyzer 24 may be an optical analyzer, such as a spectrometer. Turning to FIG. 3, the analyzer 24 includes a source of radiated energy 32 and at least one detector. The source of radiated energy 32 can be a light source. The detector can be a transmission detector 40 or a reflectance detector 38. The analyzer 24 can also include more than one detector, for example, both a reflectance detector 38 and a transmission detector 40. The source of radiated energy 32 and the detector may be selected from all available spectroscopy technologies. The analyzer 24 can also include an optical bench or a multivariate optical element (which is an optical regression calculation device) 36.

Any available spectroscopy method can be used in the determination of the at least one property of the fluid 34 or two or more properties of the fluid. The spectroscopy can be selected from the group consisting of absorption spectroscopy, fluorescence spectroscopy, X-ray spectroscopy, plasma emission spectroscopy, spark or arc (emission) spectroscopy, visible absorption spectroscopy, ultraviolet (UV) spectroscopy, infrared (IR) spectroscopy (including near-infrared (NIR) spectroscopy, mid-infrared (MIR) spectroscopy, and far-infrared (FIR) spectroscopy), Raman spectroscopy, coherent anti-Stokes Raman spectroscopy (CARS), nuclear magnetic resonance, photo emission, Mossbauer spectroscopy, acoustic spectroscopy, laser spectroscopy, Fourier transform spectroscopy, and Fourier transform infrared spectroscopy (FTIR) and combinations thereof. The exact spectroscopy method utilized may vary depending on the desired property to be determined. According to an embodiment, the spectroscopy method utilized is selected such that the desired property of the fluid 34 is capable of being detected, and preferably quantified.

The analyzer 24 can include the source of radiated energy 32. The source of radiated energy 32 can be ionizing radiation or non-ionizing radiation. The source of radiated energy 32 can be selected from the group consisting of a tunable source, a broadband source (BBS), a fiber amplified stimulated emission (ASE) source, black body radiation, enhanced black body radiation, a laser, infrared, supercontinuum radiation, frequency combined radiation, fluorescence, phosphorescence, and terahertz radiation. A tungsten-halogen light source is an example of a broadband light source for use in the Near-Infrared region that emits wavelengths ranging from 350 nm to 3,000 nm. In an embodiment, the source of radiated energy 32 includes any type of infrared source.

The source of radiated energy 32 (e.g., light) can be emitted in a desired wavelength or range of wavelengths. The desired wavelength or range can be determined based on the desired property of the fluid 34 to be determined. According to an embodiment, the desired wavelength or range of wavelengths is selected such that the at least one property of the fluid 34 can be determined. For example, if the desired property to be determined is carbon dioxide (CO₂), then the desired wavelength can be selected to be 4,300 nm as CO₂ has an absorption peak at that wavelength. The light emitted can also be in a range that encompasses the desired wavelength. For example, to detect CO₂, the light emitted can be in the mid-infrared range of approximately 2,500 to 25,000 nm. By way of another example, hydrogen sulfide gas (H₂S) can present absorption peaks at 1,900, 2,300, 2,600, 3,800 and 4,100 nm. According to this example the light emitted can include the entire IR spectrum or the NIR and MIR ranges of 800 to 2,500 nm and 2,500 to 25,000 nm, respectively. By way of another example, CH₄ and Gas-to-Oil ratio (GOR) can present absorption peaks at approximately 1,700 and 2,300 nm; whereas aromatics can present an absorption peak at approximately 2,450 nm. Accordingly, the light emitted can be in the near IR range.

According to an embodiment, the methods include the step of determining two or more properties of the fluid 34. A separate analyzer 24 (not shown in the Figures) can be used for each property to be determined. Of course an individual analyzer 24 can also be designed such that the analyzer is capable of determining two or more properties of the fluid 34. According to this embodiment, the wavelength or wavelength range can be selected such that the two or more properties of the fluid 34 can be determined. By way of example, in order to determine if both CO₂ and H₂S are present in the fluid, the wavelength range can be selected to be the MIR range of approximately 2,500 to 25,000 nm. In this manner, should CO₂ and H₂S both be present in the fluid, then absorption peaks would indicate such presence. The source of radiated energy 32 can transmit light rays in a range of from 4,000 to 5,000 nm, which is a range for absorbance of carbon dioxide. Using Beer's Law and assuming a fixed path length, the amount of carbon dioxide in the fluid 34 is proportional to the absorption of light in this range. The source of radiated energy 32 can also transmit light rays in a range of from 1,900 to 4,200 nm, which is a range for absorbance of hydrogen sulfide. Data collected from these two wavelength ranges may provide information for determining the presence and possibly the amount of carbon dioxide and hydrogen sulfide in the fluid 34. By way of another example, in order to determine if both CH₄ and aromatics are present in the fluid, the wavelength range can be selected to be the NIR range of approximately 800 to 2,500 nm.

The source of radiated energy 32 can be a light source. The light source can be in the IR range. According to an embodiment, the IR light source is a MIR range light source. In an embodiment the MIR range light source is a tunable light source. The tunable light source may be selected from the group of an optical parametric oscillator (OPO) pumped by a pulsed laser, a tunable laser diode, and a broadband source (BBS) with a tunable filter. In an embodiment, the tunable MIR light source is adapted to cause pulses of light to be emitted at or near the absorption peak of the at least one property of the fluid 34.

The water content of the fluid 34 can be determined in any manner and can be determined by optical or non-optical means. According to an embodiment, the water content in the fluid and the compensation, if any, of the optical response shifts for the determination of at least one property of the fluid can be determined.

If the tunable light source is a broadband source, then detection of the at least one property of the fluid 34 may be improved by applying frequency modulation to the broadband source signal by modulating the drive current or by chopping so that unwanted signals can be avoided in the detector of the spectrometer by using phase sensitive detection. The broadband source may be pulsed with or without frequency modulation.

In an embodiment the source of radiated energy 32 can include a laser diode array. In a laser diode array light source system, desired wavelengths are generated by individual laser diodes. The output from the laser diode sources may be controlled in order to provide signals that are arranged together or in a multiplexed fashion. By utilizing a laser diode array light source, time and/or frequency division multiplexing may be accomplished at the spectrometer. A one-shot measurement or an equivalent measurement may be accomplished with the laser diode array. A probe-type or fluid-type optical cell system may also be utilized.

The analyzer 24 can also include a sample container for the radiated energy to interact with the fluid. The sample container can be a tube or other device discussed in more detail below. The step of determining also comprises detecting the interaction between the radiated energy and the fluid 34. The detection of the interaction can occur via the use of at least one detector. The detector can be a radiation transducer. According to an embodiment, the detector is capable of detecting the interaction between the radiated energy and the fluid 34. The radiated energy can be partially or fully absorbed by the fluid 34, wherein some or none of the radiated energy is then transmitted through the fluid. According to an embodiment, the detector is capable of detecting the amount of radiated energy that is absorbed and/or transmitted by the fluid 34. The effectiveness of the detector may be dependent upon temperature conditions. Generally, as temperatures increase, the detector becomes less sensitive. The detector can include a mechanism whereby thermal noise is reduced and sensitivity to emitted radiated energy is increased. The detector can be selected from the group consisting of thermal piles, photo acoustic detectors, thermoelectric detectors, quantum dot detectors, momentum gate detectors, frequency combined detectors, high temperature solid gate detectors, and detectors enhanced by meta materials such as infinite index of refraction, and combinations thereof.

The source of radiated energy 32 can also include a splitter. For example, the light that is emitted can be split into two separate beams in which one beam passes through the fluid 34 and the other beam passes through a reference fluid. Both beams are subsequently directed to a splitter before passing to the detector. The splitter quickly alternates which of the two beams enters the detector. The two signals are then compared in order to determine the property of the fluid 34.

The spectroscopy can be performed by a diffraction grating or optical filter, which allows selection of different narrow-band wavelengths from a white light or broadband source. A broadband source can be used in conjunction with Fiber Bragg Grating (FBG). FBG includes a narrow band reflection mirror whose wavelength can be controlled by the FBG fabrication process. The broadband light source can be utilized in a fiber optic system. The fiber optic system can contain a fiber having a plurality of FBGs. Accordingly, the broadband source is effectively converted into a plurality of discrete sources having desired wavelengths.

The spectroscopy can also be Fourier spectroscopy. Fourier spectroscopy, or Fourier transform spectroscopy, is a method of measurement for collecting spectra. In Fourier transform spectroscopy, rather than allowing only one wavelength at a time to pass through the fluid to the detector, this technique lets through a beam containing many different wavelengths of light at once, and measures the total beam intensity. Next, the beam is modified to contain a different combination of wavelengths, giving a second data point. This process is repeated many times. Afterwards, a computer takes all this data and works backwards to infer how much light there is at each wavelength. The analyzer 24 can include one or more mirrors used to select the desired wavelengths to pass through the fluid 34 to the transmission detector 40. There can be a certain configuration of mirrors that allows some wavelengths to pass through but blocks others (due to wave interference). The beam can be modified for each new data point by moving one of the mirrors; this changes the set of wavelengths that can pass through. The analyzer 24 can internally generate a fixed and variable length path for the optical beam and then recombine these beams, thereby generating optical interference. The resulting signal includes summed interference pattern for all wavelengths not absorbed by the fluid.

The Fourier spectroscopy can utilize an IR light source, also referred to as Fourier transform infrared (FTIR) spectroscopy. In an embodiment, IR light is guided through an interferometer, the IR light then passes through the fluid 34, and a measured signal is then obtained, called the interferogram. In an embodiment Fourier transform is performed on this signal data, which results in a spectrum identical to that from conventional infrared spectroscopy. The benefits of FTIR include a faster measurement of a single spectrum. The measurement is faster for the FTIR because the information at all wavelengths is detected simultaneously.

According to an embodiment, the analyzer 24 is a Integrated Computational Element (ICE) calculation device. FIGS. 3-7 depict an ICE calculation device 24 according to an embodiment. Representative device 24 comprises: a light source 32; the fluid 34; an Integrated Computational Element (ICE) 36, which is an optical regression calculation device; a reflectance detector 38 for detecting light reflected from ICE 36; and a transmission detector 40 for detecting the light transmitted by ICE 36. One type of ICE is a unique optical calculation device that comprises a multiple layer optical thin-film stack.

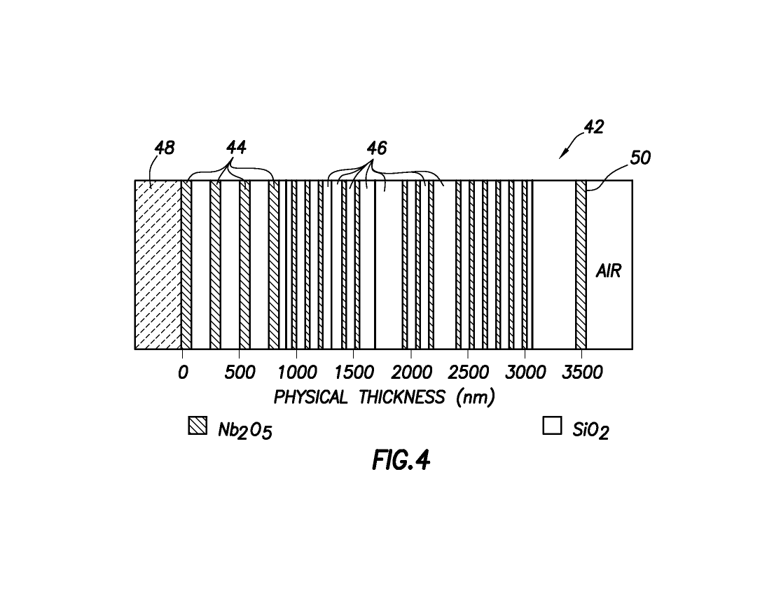

In FIG. 4, for example, representative optical regression calculating device ICE 42 comprises a plurality of alternating layers 44 and 46 respectively of Nb₂O₅ and SiO₂. The layers are deposited on an optical substrate 48, which may be of the type referred to in this art as BK-7. The other end layer 50 of the optical calculating layers can be exposed to the environment of the installation. The number of layers and the thickness of the layers are determined from, and constructed from, the spectral attributes determined from a spectroscopic analysis of a property of the fluid 34 using a conventional spectroscopic instrument.

The spectrum of interest of a given property typically comprises any number of different wavelengths. It should be understood that the ICE of FIG. 4 does not in fact represent any property of a fluid 34, such as a liquid hydrocarbon, but is provided for purposes of illustration only. The number of layers and their relative thicknesses of FIG. 4 thus bear no correlation to any fluid 34 property to which the present invention is directed and are also not to scale. The thickness of the layers may be in the order of microns each as shown.

The multiple layers can have different refractive indices. By properly selecting the materials of the layers and their spacing, the optical calculation device can be made to selectively pass predetermined fractions of light at different wavelengths. Each wavelength is given a predetermined weighting or loading factor. The various weighting factors of the ICE produce a composite signature waveform for that property. The thicknesses and spacing of the layers may be determined using a variety of approximation methods from the spectrograph of the property of interest. The approximation methods may include inverse Fourier transform (IFT) of the optical transmission spectrum and structuring the optical calculation device as the physical representation of the IFT. The approximations convert the IFT into a structure based on known materials with constant refractive indices.

The weightings that the ICE 42 layers apply at each wavelength can be set to the regression weightings described with respect to a known equation, or data, or spectral signature which can be found for the given property of interest. The optical calculation device ICE 42 performs the dot product of the input light beam from the fluid 34 and a desired loaded regression vector. The ICE 42 output light intensity, as measured by the systems optical transducer, is directly proportional to, the desired fluid 34 property to be determined.

By way of example, if the property of interest is the determination of resin in a fluid and the regression vector is used for the determination of resin, then the intensity of the light output of the ICE is proportional to the amount of resin in the fluid through which the light beam input to the optical calculation device has either passed or has been reflected from or otherwise interacted with. These wavelengths are weighted proportionately by the construct of the corresponding ICE layers. The resulting layers together produce an optical calculation device or ICE 42, which is used to modify or weight the input light intensity from the fluid 34 at each wavelength. The output light intensity measured by the optical transducer represents the summation of all of the modified wavelengths for that property, e.g., resin. The ICE output light intensity value is proportional to the amount of resin in the fluid 34 being analyzed. In this manner, an ICE is produced for each property to be determined in the fluid 34.

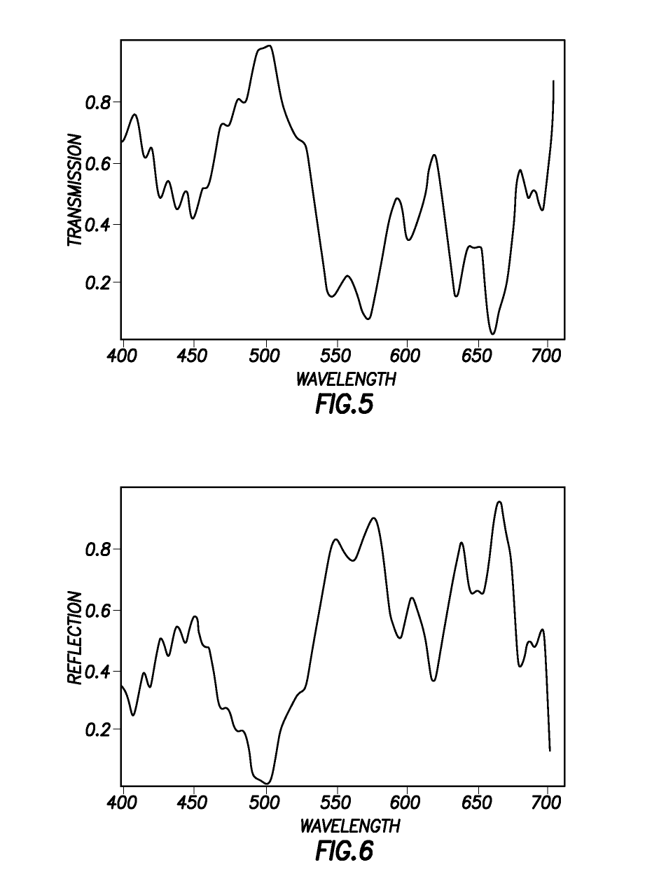

Such ICE devices represent pattern recognition devices, which produce characteristic output patterns representing a signature of the spectral elements that define the property of interest. The intensity of the light output is a measure of the proportional amount of the property in the test media being evaluated. For example, an ICE transmission and corresponding reflection output waveform, originating from the lights sources and following interaction with the fluid 34 and ICE 36, might appear as in FIGS. 5 and 6 respectively, which do not represent any specific fluid property, but are shown for purposes of illustration only. This waveform may be the light that impinges upon transmission detector 40 of FIG. 3, for example. The detectors 38/40 may be any device capable of detecting electromagnetic radiation, and may be generally characterized as an optical transducer. For example, the detectors 38/40 may be, but are not limited to, a thermal detector such as a thermopile or photoacoustic detector, a semiconductor detector, a piezo-electric detector, a charge coupled device (CCD) detector, a video or array detector, a split detector, a photon detector (such as a photomultiplier tube), photodiodes, a terahertz detector, combinations thereof, or the like, or other detectors known to those skilled in the art. Each of these detectors, such as reflectance detector 38 and transmission detector 40 associated with ICE 36, transmits its output, an electrical signal, which represents the magnitude of the intensity of the signal of FIG. 5, for example, that is incident on the detector. Thus, this signal is a summation of all of the intensities of the different wavelengths incident on the detector.

The reflected light from the ICE 36 of FIG. 3, produces a complimentary waveform of the transmitted signal of FIG. 5. The reflected light intensity is 1 minus the transmitted intensity at each wavelength. This reflected signal is represented by the waveform of FIG. 6, and is measured by reflectance detector 38. The reflected signal is subtracted from the transmitted signal of FIG. 5 by a data receiver 12 of FIG. 3, an on-board computer 106, or a remote computer. The difference (T-R) is proportional to the property in the fluid being examined. Further details regarding how ICE components are able to distinguish and process electromagnetic radiation related to the characteristic or analyze a property of interest are described in U.S. Pat. No. 6,198,531 issued to Michael L. Myrick, Matthew P. Nelson, and Karl S. Booksh on Mar. 6, 2001; U.S. Pat. No. 6,529,276 issued to Michael L. Myrick on Mar. 4, 2003; and U.S. Pat. No. 7,920,258 issued to Michael L. Myrick, Robert P. Freese, Luisa T. M. Profeta, Jonathan H. James, John C. Blackburn, and Ryan J. Priore on Apr. 5, 2011, each of which are hereby incorporated by reference in their entirety.

As can be seen in FIG. 3, the fluid 34 can be located between the light source 32 and the ICE 36. According to another embodiment, and as depicted in FIG. 7, the fluid 34 is located adjacent to the light source 32 and the ICE 36. In FIG. 7, a representative tube 80 may be part of the vessel 100, as discussed above wherein the fluid 34 can flow through the tube 80 and become analyzed during fluid flow or flow through the tube into a testing chamber wherein the fluid is analyzed, either in a static state or flowing state. The fluid 34 can be flowing in the tube 80 in direction 54. Attached to the tube 80, which may be made of a variety of materials, including stainless steel, is an ICE calculation device 24. ICE calculation device 24 corresponds to the device 24 of FIG. 3 for determining the amount of a property of the fluid 34 in the tube 80. The system utilizing the ICE calculation device 24 determines the amount of the property in real time and reports that amount instantaneously as it occurs in the fluid 34. As it is known to those in the art, there can be more than one analyzer 24 located adjacent to the tube. Additionally, there are many different configurations of analyzers 24 and tubes 80. For example, there can be a network of tubes that branch from a central input tube. Each tube of the network can include one or more analyzers 24 such that multiple properties of the fluid are analyzed.

Referring to FIG. 7, which illustrates an exemplary ICE system that uses attenuated total reflection of light from source 84 to interact with fluid 34 to determine a property of interest in fluid 34 according to one or more embodiments of the present disclosure. The ICE calculation device 24 of FIG. 7 comprises a housing 58, which may be magnetized metal or stainless steel, and a frame 60, which may be stainless steel, and which also may be magnetized and which may have appropriate protective coatings. The housing 58 and frame 60 may be circular, cylindrical, or rectangular. The housing is preferably constructed so that it is readily attachable and detachable from the tube 80. The tube 80 has a circular or rectangular opening 62 forming a window that is transparent to light, for example the IR spectral wavelengths. The housing 58 and frame 60 can be cylindrical, wherein the frame can form an internal circular opening 61.

An internal reflectance element (IRE) 64, which can be a circular, optically-transparent disc or rectangular, optically-transparent prism, or other shapes as may be used in a particular implementation, preferably of clear optically-transparent diamond, or a pair of spaced optically-transparent plates (not shown), is attached to the frame 60 in the frame opening 61 enclosing and sealing the opening 61. The IRE 64 may be bonded to the frame, for example, or attached in other ways as known in the art. The IRE 64 has two spaced parallel planar surfaces 66 and 68 and an outer annular inclined facet 70 defined by the Brewster angle, dependent upon the materials of the interface and wavelength of the light, to the surfaces 66 and 68. A Brewster angle (also known as the polarization angle) is an angle of incidence at which light with a particular polarization is perfectly transmitted through a transparent dielectric surface, with no reflection. The light source 32 is located in the housing cavity 74 and is located to cause its light 76 to be incident on the facet 70 at a right angle thereto. The facet is also at the Brewster angle or about 45° to the surface 78 of the fluid 34 in the tube 80 contiguous with the IRE surface 66. The IRE 64 and frame 60 seal the pipe opening 61 in conjunction with a gasket such as an O-ring (not shown).

Located in the cavity 74 of the housing 58 is an ICE 36 and a detector 38, 40 responsive to the output of the ICE 36 for generating an electrical intensity output signal whose value corresponds to a property of the fluid to be determined. A conductor 84 supplies power to the light source 32 and a conductor 86 receives the detector output signal. Wires such as conductor 86 may be connected to a data receiver 12, for example shown as a computer in FIG. 3, located adjacent to the analyzer 24 for determining the property of the fluid manifested by the signal on conductor 86. Alternatively, as described above, the power supply can be a battery, a local generator, or solar panels 105 could also be used to power the apparatus components.

One problem with spectroscopy of raw petroleum or “oil” is the large absorbance of crude petroleum. Crude petroleum looks black because most of the light at all visible wavelengths is absorbed even by very small amounts of the petroleum. Some crude petroleums such as condensates and some “light” oils are more transparent in the visible wavelength range, but the majority of oils are dark. Experiments have shown that path lengths through which visible light must travel to obtain an optimum signal vary from 20 to 60 micrometers (μm). Experiments have shown that for dark oils, a 40 μm path length is acceptable. For the infrared region of absorption, optimum path lengths are a little longer as crude petroleum is more transparent in this region. However up to the electromagnetic region of 2.5 μm in the infrared, path lengths are still limited to between 100 and 300 μm (0.1 to 0.3 millimeters). Crude oil or petroleum, prior to treatment, is a dirty material containing both solid particles of varying diameters and multiphase “bubbles” (water in oil, oil in water, gas in oil, or gas in water). Both the solid particles and the “bubbles” have the capacity to clog a conventional absorption spectroscopy setup in which light is passed through a set of sampling windows to a detector.

One property of light as a wave is the ability of light to change its direction at a boundary through reflection. In reflection, the angle of reflection is equal to the angle of incidence as measured from the perpendicular of the boundary surface. At a given angle whether the wave will be transmitted or reflected in the optical domain is determined by the index of refraction of the materials at the boundary as well as the angle of incidence. For a system, reflection follows the behavior that the shallower the angle of incidence, the greater the chance of reflection and the greater the difference between index of refractions, the greater the chance for reflection. For some materials index of refraction may be chosen such that total internal reflectance is achieved and all light at almost any angle will be reflected. The exception is when light hits the boundary at the Brewster angle.

Using this principle, fiber optics carry light with little transmission loss through curved paths. Because the reflection occurs at the boundary which may have a very fine transition zone (angstrom level) which acts as sharp for light with a wavelength in the visible to infrared, the reflection actually takes place in the material behind the boundary from approximately 0.3 to 5 μm. This principle has led to the development of a spectroscopic sampling technique called total internal reflectance and makes use of a device called an internal reflectance element (IRE). In this device light is passed into a material of extremely high index of refraction usually diamond or sapphire. The light bounces between two boundaries, one containing the fluid, and the other containing an optically transparent material. As light passes behind the fluid boundary some of the light interacts with the fluid as determined by normal spectroscopy. The total number of multiple reflections controlled by the element length is used to build any desired path length.

For instance at a one micrometer (μm) sampling depth, forty (40) reflections could build up a path length of forty (40) μm. Because the IRE sampling method does not suffer the constriction of a more conventional absorbance spectroscopy method, the device will not clog readily. IREs are commercially available.

In operation of the ICE calculation device 24 of FIG. 7, the light 76 from the light source 32 is transmitted by the IRE 64 to the surface 78 of the fluid 34 in the tube 80. It is known that the light 76 incident on and reflected from the fluid surface will penetrate the surface a few micrometers, e.g., 0.3-5 μm, as discussed above. It is also known, as discussed above, that penetration of light into the fluid must be to at least a depth of, or equivalent thereof, about 40 μm in order for the reflected interacted light from the fluid surface 78 to optically interact with and carry sufficient wavelength information about the fluid properties to be meaningful. Less penetration results in insufficient data being carried by the reflected interacted light to appropriately determine a property of the fluid in the tube. The total path length requirements change depending upon the fluid type, gas phase, water phase, the component being analyzed, and so on.

As a result, the light 76 from the light source 32 is reflected from the fluid surface 78 and which penetrates the surface to about 5 μm at location a. This reflected light from location a is interacted light and is reflected to the inner surface of surface 68 of the IRE 64 to produce further interacted light. Refraction indices of the diamond of the IRE 64 cause the interacted light to be reflected from the surface 68 back through the IRE to the fluid surface 78 at location b, again penetrating to a depth of about 5 μm. This reflection process is repeated at locations c and d and other locations (not shown) until an accumulated depth of about 40 μm for all of the interactions is achieved. At the last location, d in this example, the reflected interacted light from the fluid surface 78 is incident on IRE facet 70 at location 70′. Here the reflected light 82 is normal to the facet of the IRE 68 and passes through the facet 70′.

The light 82 is incident on the ICE 36 and passes through the ICE 36 to transmission detector 40. It should be understood that a reflectance detector 38 (not shown) is also responsive to reflected light from the ICE and supplied to a further conductor (not shown) and thus to the data receiver 12, such as a computer FIG. 3, as described above.

It may be desirable to determine more than one property of the fluid 34. The methods can further include the step of determining two or more properties of the fluid 34. A separate ICE calculation device 24 can be provided for each property to be determined. The IRE element 64 may have a thickness of about 1 to 2 mm and a diameter of about 10 to 20 mm when fabricated of diamond.

However, it should be understood that the distribution of light associated with the various light paths between the light source 32 and the IRE 64 is critical to the construction of the ICE 36. That is, different housings, sources attached to such housings, and IRE associated therewith all have unique light paths that may affect the light distribution. These light paths and distributions need to be taken into consideration during the ICE construction. This construction is based on a representative spectrum for the fluid property of interest. The intensities and distribution of the various wavelengths may vary from apparatus to apparatus, and thus, such light paths and distributions need to be taken into consideration in the design and construction of the ICE associated with a given apparatus.

This problem is resolved by using the housing 58, light source 32, and IRE 64 that is eventually to be utilized for the optics associated with a given ICE for use in generating the spectrum that is to be provided by a traditional spectrometer. That generated spectral data is then utilized to construct the ICE that is to be utilized with the associated housing, light, and IRE components. Thus, it is assured that the light paths and distributions from the installed housing, light source, and IRE are identical to those used to create the ICE; and thus, there will be no errors or problems in utilizing the ICE with such components. If such components ever need replacement, a new ICE needs to be constructed unique to those replacement components or otherwise compensated for changes in light distributions. Otherwise, an error in property determination may be possible if other components are utilized other than those used to create the ICE. Therefore, any components utilized in, or that may affect, the optical path lengths or wavelength distributions from light source to ICE need to be utilized to determine the spectral aspects of the property of interest used to construct the corresponding ICE. As known to those in the art, light from source 84 can interact with fluid 34 by transmission or reflection by passing through one or more windows 62 located in tube 80. Various configurations and applications of spectral elements in optical computing devices may be found in commonly owned U.S. Pat. No. 6,198,531 issued to Michael L. Myrick, Matthew P. Nelson, and Karl S. Booksh on Mar. 6, 2001; U.S. Pat. No. 6,529,276 issued to Michael L. Myrick filed on Mar. 4, 2003; U.S. Pat. No. 7,123,844 issued to Michael L. Myrick on Oct. 17, 2006; U.S. Pat. No. 7,834,999 issued to Michael L. Myrick, Jonathan H. James, John C. Blackburn, and Robert P. Freese on Nov. 16, 2010; U.S. Pat. No. 7,711,605 issued to Michael N. Santeufemia and Christopher John Moulios on May 4, 2010; U.S. Pat. No. 7,920,258 issued to Michael L. Myrick, Robert P. Freese, Luisa T. M. Profeta, Jonathan H. James, John C. Blackburn, and Ryan J. Priore on Apr. 5, 2011; U.S. Pat. No. 8,049,881 issued to Michael L. Myrick, Robert P. Freese, John C. Blackburn, and Ryan J. Priore on Nov. 1, 2011; U.S. Pat. No. 8,208,147 issued to Michael L. Myrick, Robert P. Freese, Ryan J. Priore, John C. Blackburn, Jonathan H. James, and David L. Perkins on Jun. 26, 2012; and U.S. Pat. No. 8,358,418 issued to Michael L. Myrick, Robert P. Freese, Ryan J. Priore, John C. Blackburn, Jonathan H. James, and David L. Perkins on Jan. 22, 2013, each of which are hereby incorporated by reference in their entireties.

As can be seen in FIG. 3, the step of determining at least one property of the fluid can further comprise transmitting data from the detectors 38, 40 to a data receiver 12. The data receiver 12 can directly transmit data to an operator via the transmitter and receiver module 102. Conversely, the data receiver 12 can also store the data in the data receiver 12 itself or send the data to the on-board computer 106 wherein the data is stored on the on-board computer 106 for retrieval at a later time. The data receiver 12 and/or the on-board computer 106 can be used to analyze the data from the detectors 38, 40 such that the presence of one or more properties of the fluid 34 can be determined. The data receiver 12 and/or the on-board computer 106 can also be used to quantify the amount of the one or more properties in the fluid 34. Either the raw detector data outputs may be sent to the data receiver 12 or the signals may be subtracted with an analog circuit and magnified with an operational amplifier converted to voltage and sent to the data receiver 12 as a proportional signal, for example. The raw detector outputs can also be sent to an operator via the transmitter and receiver module 102. In the case of the submersible 120, data can also be sent to an operator via a radio signal using acoustic waves at very low frequency. This can be accomplished, for example, via the antenna 104. It should be understood, that for the submersible 120, any data (e.g., data from the analyzer 24 or data regarding the course and position of the submersible) can be relayed to an operator via a radio signal or acoustic waves at very low frequency.

The methods can further include the step of retrieving the vessel from the body of water after the step of determining the at least one property of the fluid. If data is stored on the vessel, for example, in the on-board computer, then the methods can further comprise the step of retrieving, for example, downloading the data from the on-board computer. The data that is retrieved can include the data from the analyzer as well as data regarding the vessel's location. The data can be cross-referenced such that the data from the analyzer can correspond to the vessel's location at that data analysis. In this manner, an operator can use all of the data to determine the location of a reservoir, a leak, or the boundaries of an oil slick.

The methods can further comprise the step of pre-programming a desired course for the vessel 100 to move through the body of water. The step of pre-programming can include programming the course based on data received from the data receiver 12. For example, the vessel can be pre-programmed to navigate in a specific pattern until the property of interest is detected in the fluid and then the vessel is programmed to take a different course. The methods can further comprise the step of monitoring the vessel's location in the body of water. The step of monitoring the location can also comprise the step of transmitting new coordinates to the vessel.

The methods can further include the step of monitoring the data from the analyzer 24. The step of monitoring the data can be used in conjunction with the step of monitoring the location and transmitting new coordinates to the vessel. In this manner, if the property of interest is detected in the fluid, then an operator can transmit new navigation instructions to the vessel. This process can be repeated as frequently as needed to complete the operation of the vessel (e.g., a reservoir is discovered, the source of a leak in the well equipment is discovered, or the presence and/or boundaries of an oil slick are ascertained).

The sensor used to analyze the fluid in a body of water is described as being autonomous and remote. As used herein, the word “autonomous” means that the device operates independently without human intervention. For example, the analyzer is autonomous, meaning that the analyzer analyzes the fluid continuously without an operator controlling when the analyzer functions or the analyzer can be pre-programmed when to analyze the fluid, either at specific time intervals or based on data from the analyzer or the vessel's location. As used herein, the word “remote” means that the device is not physically connected to an operator's control. For example, data transmission and reception is achieved wirelessly via the transmitter and receiver module 102 and/or the antenna 104 wherein the vessel's course can be altered via the wireless communication and the communication can be transferred to the boat or submersible via the umbilical without the need for an operator to be physically present on the boat or submersible.

According to an embodiment, the analyzer is used for one or more of the following operations: detecting the presence of an oil or gas reservoir under the floor of the body of water; detecting the presence and/or location of oil or gas leaks in well equipment located at the wellhead or in the body of water; and determining the presence and/or geographic boundaries of an oil slick.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above may be altered or modified and all such variations are considered within the scope and spirit of the present invention. While compositions and methods are described in terms of “comprising,” “containing,” or “including” various components or steps, the compositions and methods also can “consist essentially of” or “consist of” the various components and steps. Whenever a numerical range with a lower limit and an upper limit is disclosed, any number and any included range falling within the range is specifically disclosed. In particular, every range of values (of the form, “from about a to about b,” or, equivalently, “from approximately a to b,”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent(s) or other documents that may be incorporated herein by reference, the definitions that are consistent with this specification should be adopted.

What is claimed is:

1. A method of analyzing a fluid in a body of water comprising:
providing a vessel, wherein the vessel moves through the body of water; and
determining at least one property of the fluid using an analyzer,
wherein the step of determining comprises: contacting the fluid with radiated energy; and detecting the interaction between the radiated energy and the fluid, wherein the analyzer is located on or adjacent to the vessel, and wherein the analyzer incorporates one or more integrated computational elements (ICE).

2. The method according to claim 1, wherein the vessel comprises a boat or a submersible.

3. The method according to claim 1, wherein the vessel further comprises at least one of the following: a navigation system; a transmitter and receiver module; an antenna; a power supply; an on-board computer; a data receiver; and a device for moving the vessel through the body of water.

4. The method according to claim 1, wherein the at least one property is selected from the group consisting of: asphaltenes; saturates; resins; aromatics; solid particulate content; hydrocarbon composition and content; gas composition C₁-C₁₃ and content; carbon dioxide gas; hydrogen sulfide gas; and correlated pressure, volume, or temperature properties including fluid compressibility, gas-to-oil ratio, bubble point, density, a petroleum formation factor, viscosity, a gas component of a gas phase of a petroleum, total stream percentage of water, gas, oil, solid particles, solid types, oil finger printing, reservoir continuity, and oil type; plankton and/or bacteria counting and typing; water elements including ion composition and content, anions, cations, salinity, organics, pH, mixing ratios, tracer components, contamination; or other hydrocarbon, gas, solids, or water properties that can be related to spectral characteristics, including the use of regression methods.

5. The method according to claim 1, wherein the determination of the at least one property of the fluid is performed using at least one of the following fluid physical properties; density, capacitance, or resistivity.

6. The method according to claim 1, wherein the analyzer comprises a source of the radiated energy, a sample container for the radiated energy to interact with the fluid, and a detector.

7. The method according to claim 6, wherein the detector is capable of detecting the interaction between the radiated energy and the fluid.

8. The method according to claim 7, wherein the step of determining the at least one property of the fluid further comprises transmitting data from the detector to a data receiver.

9. The method according to claim 6, wherein the source of the radiated energy is emitted at a desired wavelength or in a range of wavelengths.

10. The method according to claim 9, wherein the desired wavelength or range of wavelengths is selected such that the at least one property of the sample can be determined.

11. The method according to claim 1, further comprising the step of determining two or more properties of the fluid.

12. An autonomous remote sensor for analyzing a fluid in a body of water comprising:
a vessel, wherein the vessel moves through the body of water; and
an analyzer, wherein the analyzer:
(A) is located on or adjacent to the vessel;
(B) incorporates one or more Integrated Computational Elements (ICE); and
(C) is capable of determining at least one property of the fluid by at least contacting the fluid with radiated energy and detecting the interaction between the radiated energy and the fluid.

13. The autonomous remote sensor according to claim 12, wherein the vessel further comprises at least one of the following: a navigation system; a transmitter and receiver module; an antenna; a power supply; an on-board computer; a data receiver; and a device for moving the vessel through the body of water.

14. The autonomous remote sensor according to claim 12, wherein the at least one property is selected from the group consisting of: asphaltenes; saturates; resins; aromatics; solid particulate content; hydrocarbon composition and content; gas composition C₁-C₁₃ and content; carbon dioxide gas; hydrogen sulfide gas; and correlated pressure, volume, or temperature properties including fluid compressibility, gas-to-oil ratio, bubble point, density, capacitance, resistivity, a petroleum formation factor, viscosity, a gas component of a gas phase of a petroleum, total stream percentage of water, gas, oil, solid particles, solid types, oil finger printing, reservoir continuity, and oil type; plankton and/or bacteria counting and typing; water elements including ion composition and content, anions, cations, salinity, organics, pH, mixing ratios, tracer components, contamination; or other hydrocarbon, gas, solids, or water properties that can be related to spectral characteristics, including the use of regression methods.

15. The autonomous remote sensor according to claim 12, further comprising a source of the radiated energy and wherein the source of the radiated energy is emitted at a desired wavelength or in a range of wavelengths.

16. The autonomous remote sensor according to claim 15, wherein the desired wavelength or range of wavelengths is selected such that the at least one property of the fluid can be determined.

17. The autonomous remote sensor according to claim 12, wherein the analyzer is used for one or more of the following operations: detecting the presence of an oil or gas reservoir under the floor of the body of water; detecting the presence and/or location of oil or gas leaks from man-made objects in the body of water; and determining the presence and/or geographic boundaries of an oil slick in the body of water.

18. A method of analyzing a fluid in a body of water comprising:
providing a vessel, wherein the vessel moves through the body of water; and
determining at least one property of the fluid using an analyzer,
wherein the step of determining comprises: contacting the fluid with radiated energy; and detecting the interaction between the radiated energy and the fluid, wherein the analyzer is located on or adjacent to the vessel, and wherein the analyzer has a spectral resolution less than 4 nm.

19. The method according to claim 18, wherein the determination of the at least one property of the sample is performed using spectroscopy.

20. The method according to claim 19, wherein the spectroscopy is selected from the group consisting of absorption spectroscopy, fluorescence spectroscopy, X-ray spectroscopy, plasma emission spectroscopy, spark or arc (emission) spectroscopy, visible absorption spectroscopy, ultraviolet (UV) spectroscopy, infrared (IR) spectroscopy (including near-infrared (NIR) spectroscopy, mid-infrared (MIR) spectroscopy, and far-infrared (FIR) spectroscopy), Raman spectroscopy, coherent anti-Stokes Raman spectroscopy (CARS), nuclear magnetic resonance, photo emission, Mossbauer spectroscopy, acoustic spectroscopy, laser spectroscopy, Fourier transform spectroscopy, and Fourier transform infrared spectroscopy (FTIR) and combinations thereof.
